(19) United States
(12) Patent Application Publication
(10) Pub. No.: US 2008/0207578 A1
(43) Pub. Date: Aug. 28, 2008

(54) METHOD OF INDUCING APOPTOSIS IN CANCER TREATMENT BY USING CUCURBITACINS

(76) Inventors: Kee Hung Chu, Hong Kong (CN); Hongtao Xing, Hong Kong (CN)

Correspondence Address: EVAN LAW GROUP LLC, 600 WEST JACKSON BLVD., SUITE 625, CHICAGO, IL, 60661, US

(21) Appl. No.: 11/954,805

(22) Filed: Dec. 12, 2007

Related U.S. Application Data
(60) Provisional application No. 60/870,381, filed on Dec. 15, 2006.

(51) Int. Cl. A61K 31/56; A61P 35/00

(52) U.S. Cl. 514/178

(57) ABSTRACT

This invention relates to the preparation and use of anti-cancer compounds/formulation containing cucurbitacins. Said formulation comprises active ingredients, particularly cucurbitacin B and cucurbitacin D, with the efficacy of anti-proliferation and inducing cellular apoptosis. Said formulation owns the anticancer activity. This invention also provides a method of isolating and purifying the active ingredients in lab-scale and in industrial-scale.

### CROSS REFERENCE TO RELATED APPLICATION

Pursuant to 35 U.S.C. 119(e), this application claims priority to U.S. Provisional Application No. 60/870,381, entitled “Method of Inducing Apoptosis in Cancer Treatment by Using Cucurbitacins” and filed on Dec. 15, 2006, the contents of which are hereby incorporated herein by reference.

### BACKGROUND

Cucurbitacins were originally isolated as the bitter principles of the Cucurbitaceae, and were later found to be present, either non-glycosylated or glycosylated, in plants of the families Brassicaceae, Scrophulariaceae, Begoniaceae, Elaeocarpaceae, Datiscaceae, Desfontainiaceae, Polemoniaceae, Primulaceae, Rubiaceae, Sterculiaceae, Rosaceae, and Thymelaeaceae. More recently, cucurbitacins have also been isolated from several genera of mushroom, including Russula, and Hebeloma, and even shell-less marine mollusks (dorid nudibranchs) (Chen et al., 2005). A typical purification process involves extraction of the cucurbitacins in plants or plant extracts by non-polar solvents such as hexane, petroleum ether and ethanol followed by separation of cucurbitacins by column chromatography or high-performance liquid chromatography using silica gel columns (U.S. Pat. No. 5,925,356).

Traditionally, the cucurbitacins are arbitrarily divided into twelve categories, incorporating cucurbitacins A-T. The natural cucurbitacins constitute a group of diverse triterpenoid substances which are well-known for their bitterness and toxicity. Structurally, they are characterized by the tetracyclic cucurbitane nucleus skeleton, namely, the 19-(10→9β)-abeo-10 alanost-5-ene (also known as 9β-methyl-19-nor lanosta-5-ene), with a variety of oxygenation functionalities at different positions. They are present in many plants as β-glucosides and function as an allomone to protect the plants from herbivores (Setzer et al., 2003). Recently, cucurbitacins are also known to possess a number of potent pharmacological effects, deriving largely from their cytotoxic, anti-cancer and anti-inflammatory properties.

Several authors have reviewed that the pharmacology activities of the several plant species are found dominantly contributed from cucurbitacins. For example, the isolation of cucurbitacin B from Picrorhiza scrophulariaeflora inhibits mitogen-induced T cell proliferation (Smit et al., 2000). Besides, cucurbitacin B and isocucurbitacin β isolated from Helicteres Isora, Ipomopsis Aggregata or Casearia arborea has been found to have cytotoxic activity against Eagle's carcinoma of the nasopharynx in cell culture (Bean et al., 1985), human nasopharyngeal carcinoma (Arisawa et al., 1984) and National Cancer Institute (NCl) 60-cell lines of human tumor screening (Beutler et al., 2000), respectively. Cucurbitacin B, and two new cucurbitane triterpenoids, leucopaxillones A and B, isolated from Leucopaxillus gentianeus have been found to inhibit the proliferation of human lung carcinoma, epatoblastoma, breast adenocarcinoma, and kidney carcinoma cell lines (Clericuzio et al., 2004). A new cucurbitacin D analogue, 2-deoxycucurbitacin D, cucurbitacin D and 25-acetylcucurbitacin F isolated from Sloanea zuliaensis have demonstrated cytotoxic activity against breast, lung and central nervous system human cancer cell lines (Rodriguez et al., 2003). Cucurbitacin D, E and I found in Gonystylus Keithii have been shown to be cytotoxic toward renal tumor, brain tumor and melanoma cell lines (Fuller et al., 1994). Cucurbitacin E purified from Conobea Scoparioided has been demonstrated to have inhibitory effect towards leukocyte intergrin-mediated cell adhesion (Musza et. al., 1994). Elaeocarpus dolichostylus has been found to have cytotoxic activity towards nasopharynx carcinoma cell lines, given cucurbitacin F being isolated by bioactivity-directed fractionation (Fang et al., 1984); and cucurbitane triterpenoids, cayaponosides B, B3, D, D3b and C2 isolated from Cayaponia tayuya have exhibited inhibitory effect on Epstein-Barr virus activation (Konoshima et al., 1995).

Cucurbitacin B has been found in many Cucurbitaceae species. It has been shown to stimulate feeding by spotted cucumber and diacroctic beetles and acts as an antigibberellin (Arisawa et al., 1984). Regarding the antitumor activity, cucurbitacin B has been shown in earlier work to demonstrate significant inhibitory activity against cultured KB cells (Oberlies et al., 2001). In addition, cucurbitacin B has exhibited anti-inflammatory activity in acetic acid-induced vascular permeability, serotonin-induced paw edema, and bradykinin-induced paw edema in mice (Yesilada et al., 1989). It has significant anti-inflammatory activity, preventive and curative effects against CCl₄-induced hepatotoxicity (Ahmad et al., 1999), and has sub-micromolar potency in the screening of natural products as antagonists of CD 18-mediated cell adhesion (Musza et al., 1994). Recent research has also shown that cucurbitacin B has potent inhibitory activity on selected gene expression in human osteoblast-like cells (Chen et al., 2005).

In contrast to cucurbitacin B, cucurbitacin D, which lacks the acetyl group at the 25-OH, is the most ubiquitous cucurbitacin known. It has also been found to antagonize the action of insect steroid hormones, and interfere with the growth of symbiotic bacteria of entomopathogenic nematodes in vitro (Barbercheck et al., 1996). Cucurbitacin D has showed significant cytotoxicity against a variety of human cancer cell lines from many independent studies, including lung cancer, human colon cancer, human oral epidermoid carcinoma, hormone-dependent human prostate cancer, human telomerase reverse transcriptase-retinal pigment epithelial cells, and human umbilical vein endothelial cells, breast (MCF-7), as well as, central nervous system (SF-268) cancer cell lines (Chen et al., 2005). It has also been found that cucurbitacin D was able to induce morphological changes of Ehrlich ascites tumor cells at low concentrations and to affect respiration, permeability, and viability of these cells at higher concentration (Duncan et al., 1996). Furthermore, cucurbitacin D has been shown to enhance capillary permeability, which is then associated with a persistent fall in blood pressure and the accumulation of fluid in thoracic and abdominal cavities in mice (Edery et al., 1961).

The cell cycle is a collection of highly ordered processes that results in the duplication of a cell. As cells progress through the cell cycle, they undergo several discrete transitions. There are four stages, G1-S-G2-M, in one cell cycle. In response to mitogenic signals, cells progress from the resting phase, G0, to G1 during which they become committed to progression through the cell cycle. From G1 they enter S phase when DNA replication occurs. After a second gap or growth phase, G2, they enter mitosis (M) when nuclear (separation of chromosomes) and cytoplasmic (cytokinesis) division occur (Elledge, 1996).

A member of the family of the cyclin dependent protein kinases (CDKs) initiates each stage of the transitions. In response to the interaction of mitogenic factors with their receptors at the outside of the cell, signaling to the nucleus results in expression of the D-type cyclins which associate with and activate the kinases CDK4 and CDK6. These CDK complexes phosphorylate the tumor suppressor protein, phosphorylated retinoblastoma (Rb), and promote the expression of cyclin E, via a mechanism that is not completely understood. Cyclin E associates with CDK2 to drive cells from G1 to S phase through phosphorylation of a limited number of targets including pRb. On entry into S phase, cyclin E is abruptly destroyed by the proteasome to which it is targeted by ubiquitination. Cyclin A, expressed in response to the CDK2/cyclin E activities, then associates with CDK2 to drive cells through S phase. CDK2/cyclin A phosphorylates a large number of target proteins including pRb, transcription factors, regulators of transcription factors and pre-replication complexes. Cyclin A remains present throughout G2 and associates with CDK1 during the transition from G2 to M phase after which it is abruptly degraded. Cyclin B then associates with CDK1 to drive cells through M phase in concert with other kinases and down stream regulatory enzymes (Johnson et al., 2002).

The CDKs are tightly regulated. In addition to the dependence on the association with cyclin, CDKs require activatory phosphorylation (on Thr160 in CDK2) by a cyclin dependent activatory kinase (CAK or CDK7/cyclin H) in the region of the kinase termed the activation segment. CDK activity may be inhibited by two distinct mechanisms, in which Ink4 and Cip1/Kip1 are inhibited or the glycine rich loop is phosphorylated by Wee1 and Myt1 kinases. The inhibitory phosphorylations are relieved by the action of the phosphatase Cdc25C, which in turn is subjected to regulation and provides an important checkpoint control, especially in response to DNA damage. p53 is a protein that functions to block the cell cycle and induce apoptosis if the DNA is severely damaged. If the damage is severe this protein can cause apoptosis (cell death). p27 is a protein that binds to cyclin and CDK blocking entry into S phase (Morgan, 1997; Johnson et al., 2005).

U.S. Pat. No. 5,925,356 provides a method of isolating and purifying cucurbitacins. The method involves the production of a cucurbitacin-containing liquid from the plant matter containing cucurbitacins. The liquid is then sequentially extracted with a non-polar solvent and then a moderately polar solvent. In a preferred embodiment, the cucurbitacin is purified by flash-column chromatography.

W.O. Pat. No. WO 02/078617 describes the treatment of tumors and cancerous tissues and the prevention of tumorigenesis and malignant transformation through the modulation of JAK/STAT3 intracellular signaling. The pharmaceutical compositions contain cucurbitacin I, or a pharmaceutically acceptable salt or analog thereof, was applied to a patient, wherein the tumor is characterized by the constitutive activation of the JAK/STAT3 intracellular signaling pathway. It also illustrates the methods of moderating the JAK and/or STAT3 signaling pathways in vitro or in vivo using cucurbitacin I, or a pharmaceutically acceptable salt or analog thereof. Another aspect of the present invention concerns a method for screening candidate compounds for JAK and/or STAT3 inhibition and anti-tumor activity.

### SUMMARY

The subject invention relates to the discovery of cucurbitacins owing the anti-cancer activity via the activation of apoptosis and regulation of MAPK signaling pathway, as well as the extraction and purification method of cucurbitacins from Cucurbitaceaes. The subject invention pertains pharmaceutical compositions containing cucurbitacin B, cucurbitacin D and/or a pharmaceutically acceptable analog thereof, that induce cytotoxic effect, cell cycle arrest, inhibit tumor cell proliferation and/or promote apoptosis thereof in human cancer cell lines. The subject invention concerns the evidence that cucurbitacin B and/or cucurbitacin D promote apoptosis by the activation of the apoptotic inducer, PARP. The subject invention also demonstrates the cucurbitacin B and/or cucurbitacin D induce cell cycle arrest by the activation of ERK, down-regulation of cyclin E, phosphorylated retinoblastoma and c-myc.

### BRIEF DESCRIPTION OF THE FIGURES

FIG. 1 shows the chemical structures, formula and mass of cucurbitacin analogs, including cucurbitacin A, B, C, D, E, F, H, I, J, L, O, P, Q and S.

FIG. 2 is a schematic diagram of process steps representative of an embodiment of the present invention in which the pure cucurbitacins are extracted and isolated from the plant material.

FIG. 3 is a table representative of an embodiment of the present invention showing the summary of growth inhibition effect of cucurbitacin B and cucurbitacin D on 59 cell lines.

FIG. 4 is a graph representative of an embodiment of the present invention in which the growth inhibition effect of cucurbitacin B and cucurbitacin D on 9 cancer groups is listed.

FIG. 5 is a table representative of an embodiment of the present invention in which the growth inhibition effect of cucurbitacin B and cucurbitacin D on selected cell lines for cell cycle analysis is illustrated.

FIG. 6 is a table representative of an embodiment of the present invention indicating the summary of effect of cucurbitacin B on cell cycle in nine cancer groups.

FIG. 7 is a table representative of an embodiment of the present invention showing the summary of effect of cucurbitacin D on cell cycle in nine cancer groups.

FIG. 8 is a graph representative of an embodiment of the present invention in which the flow cytometric analysis of cell cycle on HL60 (TB) cells treated by cucurbitacin B is illustrated.

FIG. 9 is a graph representative of an embodiment of the present invention in which the flow cytometric analysis of cell cycle on SF-295 cells treated by cucurbitacin D is demonstrated.

FIG. 10 is a table representative of an embodiment of the present invention illustrating the summary of inducing effect of cucurbitacin B on apoptosis in nine cancer groups.

FIG. 11 is a graph representative of an embodiment of the present invention in which the flow cytometric analysis of apoptosis on TK-10 cells treated by cucurbitacin B is shown.

FIG. 12 is a table representative of an embodiment of the present invention demonstrating the summary of inducing effect of cucurbitacin D on apoptosis in nine cancer groups.

FIG. 13 is a graph representative of an embodiment of the present invention in which the flow cytometric analysis of apoptosis on U251 cells treated by cucurbitacin D is indicated.

FIG. 14 is a picture representative of an embodiment of the present invention in which the cleavage of PARP with cucurbitacin B or cucurbitacin D treatment on HL60 cell lines is illustrated.

FIG. 15 is a picture representative of an embodiment of the present invention in which the western blot analysis of phosphorylated-ERK, ERK, cyclin E, phosphorylated-Rb, Rb and c-myc in HL60 cells lysates treated with cucurbitacin B or cucurbitacin D is demonstrated.

FIG. 16 is four graphs representative of an embodiment of the present invention indicating the relative expression of (A) phosphorylated-ERK; (B) cyclin E; (C) phosphorylated retinoblastoma and (D) c-myc in HL60 cells treated with cucurbitacin B and cucurbitacin D.

### DETAILED DESCRIPTION

### Definitions

The following definitions are used throughout the application.

As used herein, the term “extract(s)” denotes all possible extracts from Cucurbitaceae family, for example, but not limited to, Trichosanthes, Cucurbita pepo, Cucumis sativus and Citrullus ecirrhosus, which are obtained during the sample preparation process regardless of solvent and conditions.

As used herein, the term “ingredient(s)” denotes all possible products that are obtained during the sample purification process and contains lead compounds from herbs Cucurbitaceaes.

As used herein, the term “compound(s)” denotes all possible lead compounds, either isolated from natural products or chemically synthesized, responsible for the biomedical activity motioned in this patent.

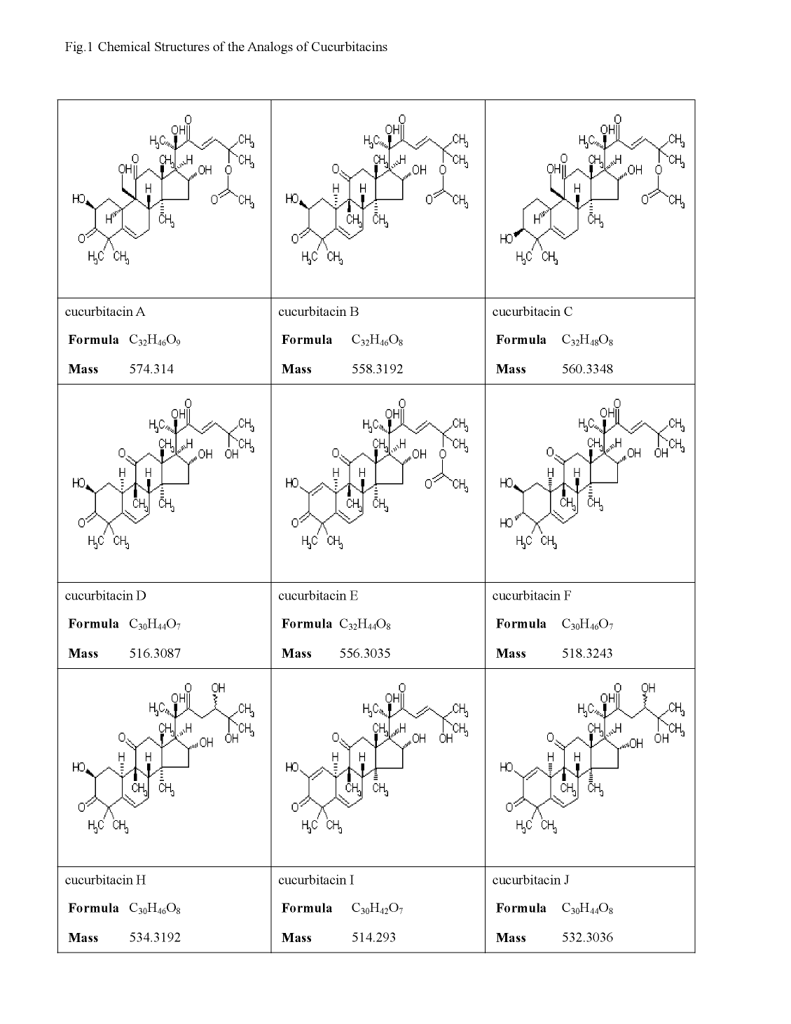

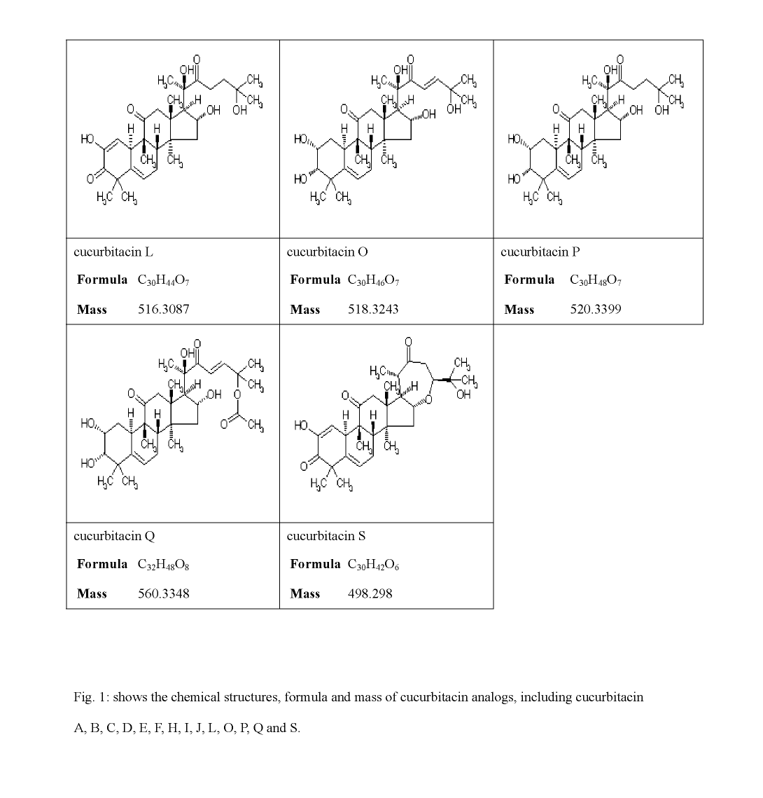

As used herein, the term “cucurbitacins” denotes all the chemical analogs of cucurbitacin, for example, but not limited to, cucurbitacin A, cucurbitacin B, cucurbitacin C, cucurbitacin D, cucurbitacin E, cucurbitacin F, cucurbitacin H, cucurbitacin I, cucurbitacin J, cucurbitacin L, cucurbitacin 0, cucurbitacin P, cucurbitacin Q and cucurbitacin S as shown in FIG. 1.

As used herein, the term “formulation(s)” denotes all possible formulations that consist either single or combined ingredients of the traditional herbal medicine Cucurbitaceae, and/or extracts derived thereof, and/or ingredients isolated thereof, and/or compound(s) purified thereof.

As used herein, the term “Trichosanthes” denotes any species of the Trichosanthes genus. Examples of such plant include, but are not limited to, Trichosanthes kirilowii Maxim, Trichosanthes rosthornii Harms, Trichosanthes japonica Regel.

As used herein, the terms “Cucurbitaceae” and “Trichosanthes” also denote any constituent of the herbal plant. Examples of such constituents include, but are not limited to, root, stem, leaf, flower, fruit and seed.

As used herein, the term “L” means liters, “mL” means milliliters, “μl” means microliter, “g” means grams, “mg” means milligram, “ng” means nanograms, and all temperatures are in degrees Celsius.

As used herein, the term “cancer” denotes any type of malignant disease characterized by neoplastic, tumorigenic or malignant cell growth. Examples of thalassemia include, but are not limited to, prostate cancer, breast cancer, lung cancer, liver cancer, colon cancer, gastric cancer, and leukemia.

As used herein, the term “non-polar” means any organic solvent with a polarity index (Snyder 1978) of not greater than 2.0, and preferably not greater than 1.6. Examples of such non-polar solvents include, but are not limited to, hexane, petroleum ether, carbon tetrachloride, and mixtures of any solvents with the specified polarity index.

As used herein, the term “polar” means any organic solvent with a polarity index (Snyder 1978) of greater than 2.0, and preferably greater than 4.0, and generally easily miscible with water. Examples of such moderately polar solvent include, but are not limited to, methanol, ethanol, acetonitrile, and mixtures of any solvents with the specified polarity index.

### Natural Products Resource

Cucurbitacins are group of complex compounds found in plants of Cucurbitaceae family. They are responsible for the bitter taste in the eggplant or cucumber. The common cucurbitacins identified including cucurbitacins A, cucurbitacins B, cucurbitacins D, cucurbitacins E, cucurbitacins I and cucurbitacins Q. Cucurbitaceaes that are rich in cucurbitacins, include Trichosanthe, Cucurbita pepo, Cucumis sativus and Citrullus ecirrhosus. Other herb like Picrorhiza kurroa from the Scrophulariaceae family (Stuppner and Wagner, 1989) or Iberis umbellate from the Cruciferae family (Dinan, 1997) are also rich in cucurbitacins. Cucurbitacins can be distributed among the whole plant, but usually more abundant in the seeds and fruits of the plant. Some members of Cucurbitaceae which are rich in cucurbitacins have been used as traditional herbal medicines for a long history. For example, Trichosanthes has been widely prescribed by herbal practitioners for thousands of years in China. The earliest record of Trichosanthes application was recorded by the ancient emperors Huangdi and Shennong 5000 years ago in “Shennong ben cao jing”, which is the herbal medicine handbook marking the classical pharmacopoeia of the heavenly husbandman.

### Cucurbitacins Extraction and Isolation

This invention provides a method for extracting, and purifying cucurbitacins from Cucurbitaceaes.

Accordingly, a first aspect of this invention provides a method for extracting fractions that contains cucurbitacins from the plant tissues of Cucurbitaceaes, for example, but not limited to, Trichosanthes. Either water or organic solvents, preferably their mixture, can be used to prepare the extract.

A second aspect of the invention provides a method for isolating active ingredients from herb Cucurbitaceaes, for example, but not limited to, Trichosanthes. Fractionally isolated ingredients can be prepared according to different purification procedures. Examples of such procedures include, but are not limited to, rotary evaporation, organic solvents extraction, centrifugation, solid phase extraction, chromatography and etc.

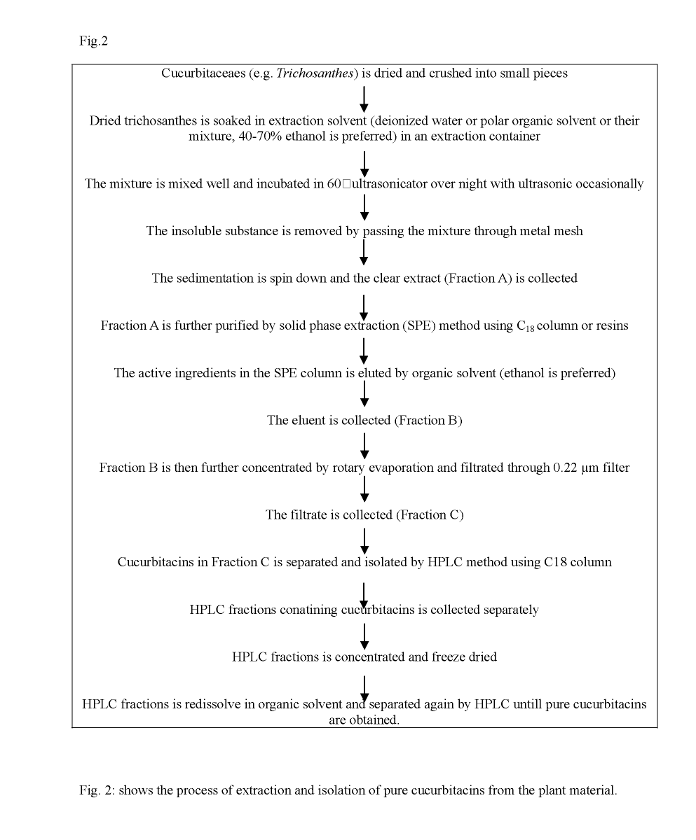

Referring to FIG. 2, the original plant materials may be sliced, dried, or physically disintegrated prior to processing. The extraction of herbal plant Cucurbitaceaes, for example, but not limited to Trichosanthes, may be obtained by any method known in the art, but preferably obtained by soaking the dried plant tissues in water or polar organic solvents or their mixture at any ratio. Such mixture should be enclosed and incubated at a certain temperature, which is usually, but not limited to, ranges between the room temperature and boiling temperature of the solvent. Resulting extract contains biological active ingredients and compounds in liquid phase. The liquid phase is isolated from the remaining insoluble materials by any means known in the art, but preferably by filtrating through medical gauze. Remaining insoluble materials may be further removed by centrifugation. The resulting liquid (Fraction A) is typically clear and additional filtration will be performed if necessary. The previous obtained Fraction A can be optionally further concentrated into a viscous liquid phase by any means known in the art, preferably by rotary evaporation. Fraction A can also be optionally extracted with a non-polar solvent to remove those essentially produced contaminants as pigments, lipids, fatty acids and waxes from aqueous phase.

Further purified ingredients can be obtained if Fraction A is processed by subsequent separation methods. Examples of such methods include, but not limited to, liquid-liquid extraction, solid phase extraction (SPE), super filtration, super critical extraction and etc. For liquid-liquid extraction, a polar organic solvent is always provided to extract a mixture of partially purified ingredients. For SPE, the column is generally eluted by a first polar organic solvent to remove the irrelative ingredients, and then eluted by a second polar organic solvent, usually with less polarity index, to wash out ingredient comprising the active compounds. Finally the second elution solvent is collected (Fraction C). This Fraction C is then further concentrated by rotary evaporation and filtrated through 0.22 μm filter (Fraction D).

The cucurbitacins in Fraction D can be isolated by further separation methods. Examples of such methods include, but not limited to, thin layer chromatography (TLC), gas chromatography (GC), liquid chromatography (LC), and high-performance liquid chromatography (HPLC), of which HPLC is preferred. Different columns can be adopted during HPLC purification. Examples of such columns include, but not limited to, normal phase columns, reverse phase columns, ion-exchange columns, and size-exclusion columns, of which C₁₈ reverse phase columns are preferred.

### Biomedical Applications

Cucurbitacins are a group of highly structurally diverse triterpenes, with a rich variety of side chain derivatives and different pharmacological activities. Although the cytotoxicity of cucurbitacin B and D was known before 1800 AD, very little is known about the mechanism of the effect of cucurbitacin B and D at the cellular and molecular level, which accounts for the relatively slow advance in cucurbitacin based anti-cancer drug discovery. With more structurally diverse cucurbitacin-related compounds being isolated and characterized from natural sources, coupled with the advance of molecular pharmacology of cancer and inflammatory diseases, which allows activities to be assayed rapidly and molecular mechanisms deciphered, it can be anticipated that cucurbitacins could be used as templates for anti-inflammatory and anti-cancer drug discovery.

### EXAMPLE 1

### Laboratory-Scale Preparation of Cucurbitacin B and Cucurbitacin D

A) Crude Extract

One kilogram of cucurbitacin-containing plant, Trichosanthes, is crushed into small pieces and oven dried. Deionized water or polar organic solvent or their mixture, 30-60% ethanol is preferred, is added into the Trichosanthes for extraction in a 5 L bottle (ratio approximately: 1 kg herb:4 L extraction solvent). The mixture is mixed well and incubated in a 60° C. ultrasonicator over night with sonication occasionally. Then the insoluble substance is removed by passing the mixture through a cheese cloth. Then the sedimentation is spun down and clear fitrate is collected.

B) Solid Phase Purification

The extract from section A is further purified by solid phase extraction method using C₁₈ column. The extract is firstly loaded into the absorbent and the cucurbitacins are eluted by organic solvent (ethanol is preferred). The cucurbitacin-containing eluent is collected in sample collection tube. The eluent is then rotary evaporated to a small volume. An organic solvent (ethanol is preferred) is added into the eluent until a clear solution obtained.

C) First HPLC Purification

The herbal extract from section B is then purified by HPLC technique using C₁₈ column. It is firstly purified by a Waters© Atlantis d C₁₈ column using acetonitrile and water as mobile phase. The fraction containing cucurbitacin B and cucurbitacin D is collected.

D1) Purification of Cucurbitacin B

The fraction containing cucurbitacin B from section C is then purified by Waters© Symmetry Prep C₁₈ column using methanol and water as mobile phase and the fraction containing cucurbitacin B is collected. The collected fraction is then purified again by Waters© Symmetry Prep C₁₈ column using acetonitrile and water as mobile phase to obtain pure cucurbitacin B.

D2) Purification of Cucurbitacin D

The fraction containing cucurbitacin D from section C is then purified by Waters© Symmetry Prep C₁₈ column using methanol and water as mobile phase and the fraction containing cucurbitacin D is collected. The collected fraction is then purified again by Waters© Symmetry Prep C₁₈ column using acetonitrile and water as mobile phase and the fraction containing cucurbitacin D is collected. The collected fraction is finally purified by a Waters© Atlantis d C₁₈ column using methanol and water as mobile phase to obtain pure cucurbitacin D.

### EXAMPLE 2

### Large-Scale Preparation of Cucurbitacin B and Cucurbitacin D

A) Crude Extract

Twenty kilograms of cucurbitacin-containing plant, Trichosanthes, are crushed into small pieces and oven dried. Deionized water or polar organic solvent or their mixture, 30-70% ethanol is preferred, is added into the Trichosanthes for extraction in a 100 L reaction tank (ratio approximately: 1 kg herb: 4 L extraction solvent). The mixture is mixed well and incubated in a 60^{˜} with constant stirring. The insoluble substance is removed by passing the mixture through a metallic mesh. Then the extract is allowed to settle at room temperature for overnight and the upper clear solution is obtained.

C) Solid Phase Purification

The extract is subjected to pass through resins, for example, DM11, and cucurbitacins adhered on the resins are eluted by organic solvent (ethanol is preferred). The eluent is concentrated and adjust to ethanol content below or equal to 40%. It is then purified by solid phase extraction method using C₁₈ column. The extract is loaded into the absorbent and cucurbitacins are eluted by organic solvent (ethanol is preferred). The cucurbitacin-containing eluent is collected in sample collection vessel. The eluent is then rotary evaporated to a small volume. An organic solvent (ethanol is preferred) is added into the eluent until a clear solution obtained.

C) First HPLC Purification

The herbs extract from section B is then purified by preparative HPLC technique using C₁₈ columns. It is firstly purified by a Waters© Atlantis Prep d C₁₈ column using ethanol and water as mobile phase. The fraction containing cucurbitacin B and cucurbitacin D is collected.

D1) Purification of Cucurbitacin B

The fraction containing cucurbitacin B from section C is then purified by Waters© Symmetry Prep C₁₈ column using methanol and water as mobile phase and the fraction containing cucurbitacin B is collected. The collected fraction is then purified again by Waters© Symmetry Prep C₁₈ column using acetonitrile and water as mobile phase to obtain pure cucurbitacin B.

D2) Purification of Cucurbitacin D

The fraction containing cucurbitacin D from section C is then purified by Waters© Symmetry Prep C₁₈ column using methanol and water as mobile phase and the fraction containing cucurbitacin D is collected. The collected fraction is finally purified by a Waters© Atlantis d C₁₈ column using methanol and water as mobile phase to obtain pure cucurbitacin D.

### EXAMPLE 3

### Cucurbitacin B and Cucurbitacin D Produced Cytostatic Effect on Human Cancer Cell Lines

Human cancer cell lines from the 59-NCl Cancer Cell Line Panel including leukemia, melanoma and cancers of breast, brain, colon, lung, ovary, prostate and kidney, were purchased from the National Institute of Cancer (USA). The growth inhibition (GI₅₀) of these cell lines treated with cucurbitacin B and cucurbitacin D in various concentrations was investigated.

59 human cancer cell lines were maintained in RPMI 1640 (Invitrogen Life Technologies, Calif., USA) supplemented with 5% Fetal Bovine Serum, 2 mM L-glutamine and 1% Penicillin/Streptomycin (Invitrogen Life Technologies) at 37° C. with 5% CO₂. All chemicals were purchased from Sigma (St. Louis, Mo., USA) unless specified otherwise.

Sulforhodamine B (SRB) assay was applied to determine the cytostatic effect of cucurbitacin B and cucurbitacin D. SRB is a dye which binds to cellular proteins and will be dissolved in base. The biomass of total protein can be measured at 520 nm using a plate reader.

Cells were inoculated into 96-well microtiter plates including “Time zero” (Tz) plates in 100 μl at cell concentrations from 5000 to 40,000 cells per well according to NCI guideline. The cells were incubated at 37° C. with 5% CO₂ for 24 hours. Cucurbitacin B and cucurbitacin D were added to the cells of final concentrations ranging from 0.488 to 4000 nM for 48 hours at 37° C. with 5% CO₂. Cold trichloroacetic acid (TCA) was added at final concentration of 10% (w/v) to adherent cells and 16% w/v to suspension cells for cell fixation for at least 60 minutes at 4° C. The supernatant was discarded and the plates were washed in tap water for 5 times and air dried. 0.4% (w/v) SRB solution was added to stain the cells for 10 minutes at room temperature. The plates were then washed with 1% acetic acid (Merck, Darmstadt, Germany) for 5 times and air dried. Bound SRB was solubilized with 100-200 ul per well of 10 mM trizma base and absorbance was measured at a wavelength of 520 nm. The percentage of growth inhibition was calculated as follows:

% of growth inhibition=100−{[(Ti−Tz)/(C−Tz)]×100}

Where:

Ti=Corrected absorbance of treatment well

Tz=Corrected absorbance of time zero well

C=Corrected absorbance of control well

The growth inhibition of 50% (GI₅₀) was obtained from the dose response curve of percentage of inhibition against dosage.

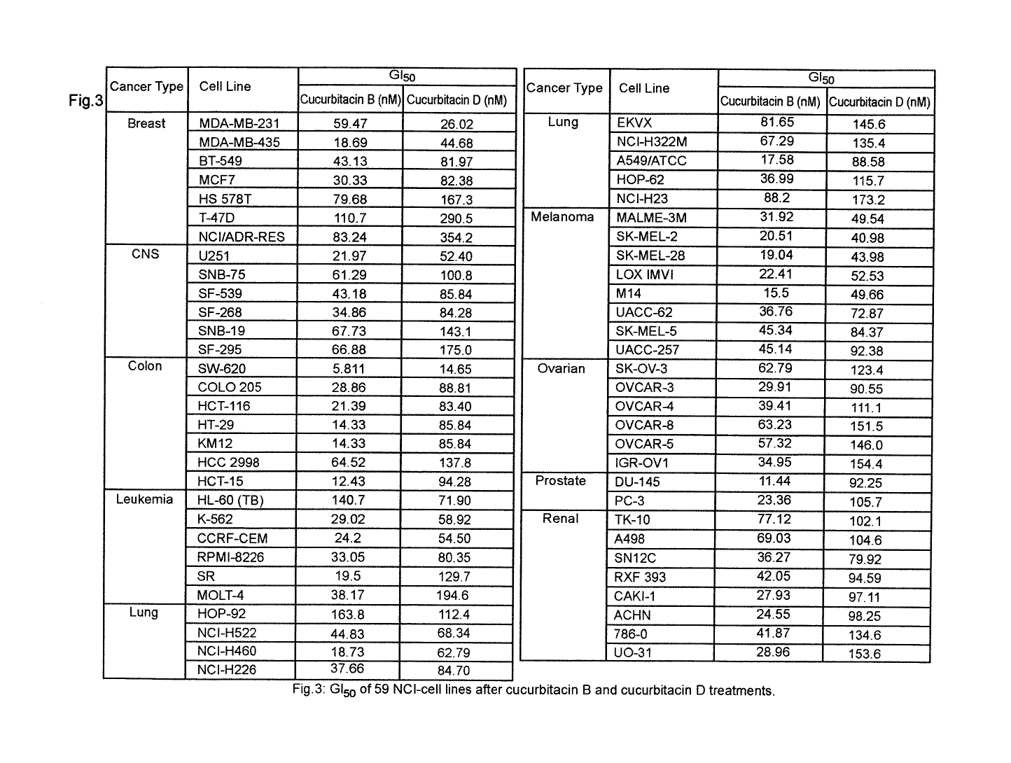

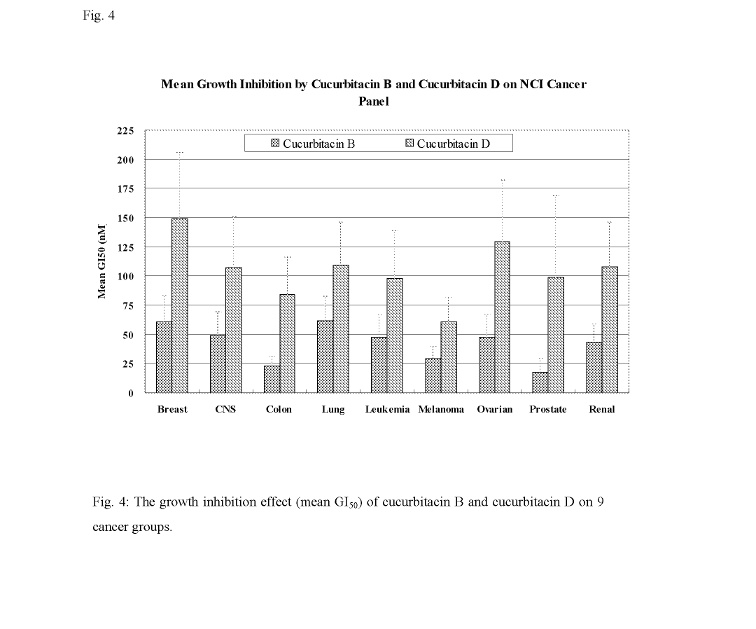

Both cucurbitacin B and cucurbitacin D inhibited the growth of 59 cancer cell lines dose-dependently. Different cell lines response differently to the two compounds. For cucurbitacin B, GI₅₀ varies from 5.8 to 164 nM, which is much lower than those treated with cucurbitacin D. Prostate cancer is the most sensitive type of cancer when treated with cucurbitacin B (mean GI₅₀=17 nM). For cucurbitacin D, the GI₅₀ varies from 14 to 354 nM while melanoma is the most sensitive cancer type (mean GI₅₀=60 nM). (FIGS. 3 and 4)

### EXAMPLE 4

### Cucurbitacin B and Cucurbitacin D Produced Cell Cycle Arrest on Human Cancer Cell Lines

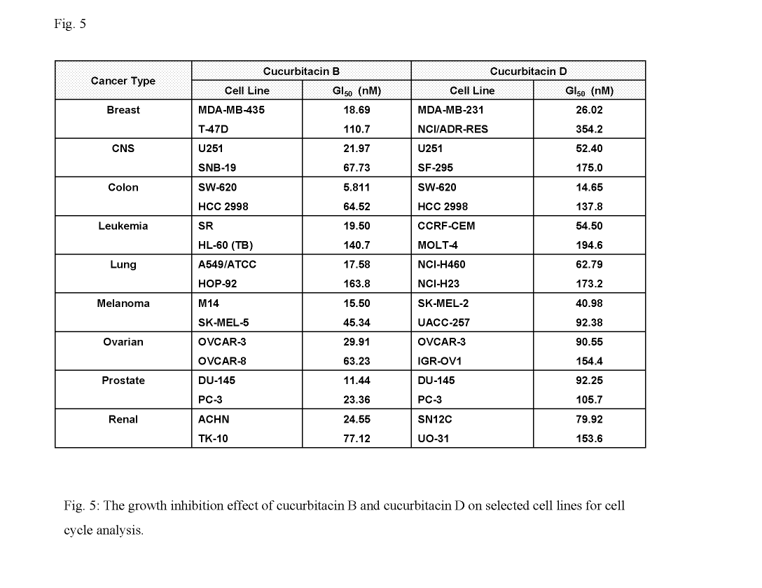

Mutation causes the cancer cells to proliferate unrestrictedly. It may result from abnormal cell cycle control. Human cancer cell lines from the 59-NCl Cancer Cell Line Panel including leukemia, melanoma and cancers of breast, brain, colon, lung, ovary, prostate and kidney were purchased from the National Institute of Cancer. Cell lines which possess the highest or lowest sensitivity (according to GI₅₀) in response to cucurbitacin B and cucurbitacin D were selected (FIG. 5). They were treated with cucurbitacin B and cucurbitacin D in three different concentrations according to the GI₅₀ to elucidate the ability to cause any changes in cell cycle.

Cancer cell lines were maintained in RPMI 1640 supplemented with 5% Fetal Bovine Serum, 2 mM L-glutamine and 1% Penicillin/Streptomycin at 37° C. with 5% CO₂. All chemicals were purchased from Sigma unless specified otherwise.

Cells were inoculated into tissue culture flask at cell concentrations from 50000 to 400,000 cells/ml according to the NCl guideline. The cells were incubated at 37° C. with 5% CO₂ for 24 hours. Cucurbitacin B and cucurbitacin D were added to the cells of final concentrations ranging from 6 to 350 nM (GI₅₀, ½ GI₅₀ and ¼ GI₅₁) of particular cell line) for 48 hours at 37° C. with 5% CO₂. Cells were then harvested and fixed in 80% cold ethanol for 30 minutes at −20° C. The ethanol was removed by centrifugation. 500 μl of PI/RNase solution (10 μg/ml propidium iodine and 300 μg/ml RNase) (Becton Dickinson, Calif., USA) was added to stain the cells which were incubated at room temperature for 15 min and filtered with 53 μm nylon mesh. Fifteen thousands cell cycle events were collected by the FACS caliber (Becton Dickinson) and the cell cycle distribution was analyzed by the ModFit™ LT software (Becton Dickinson).

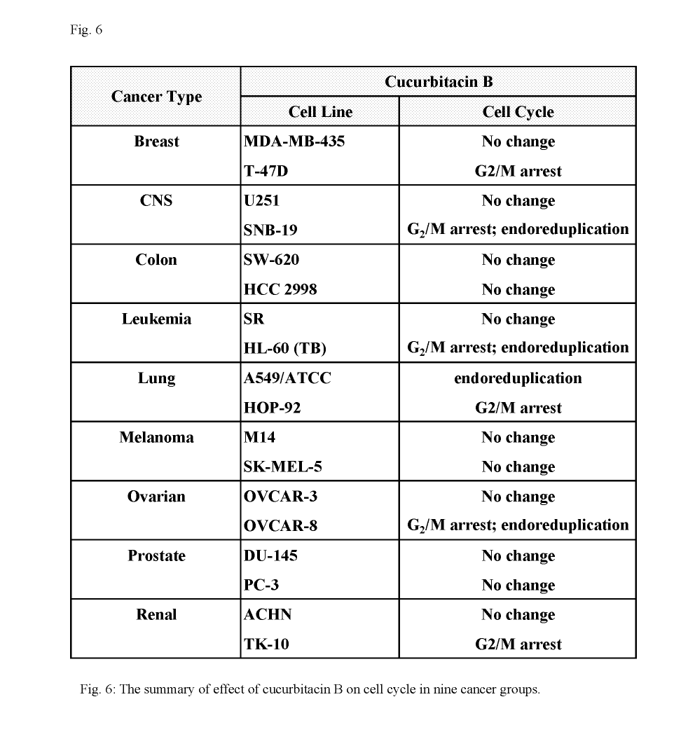

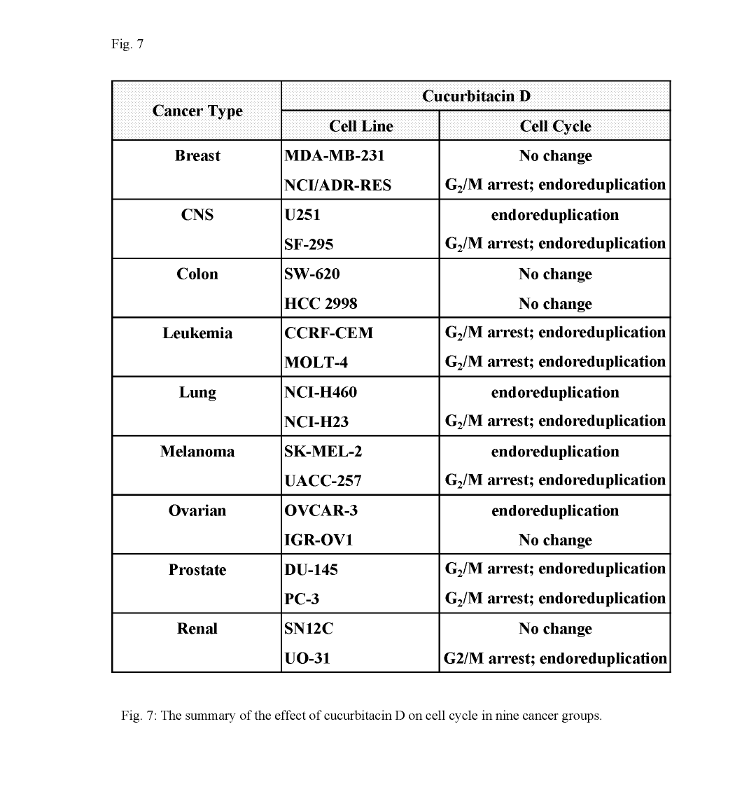

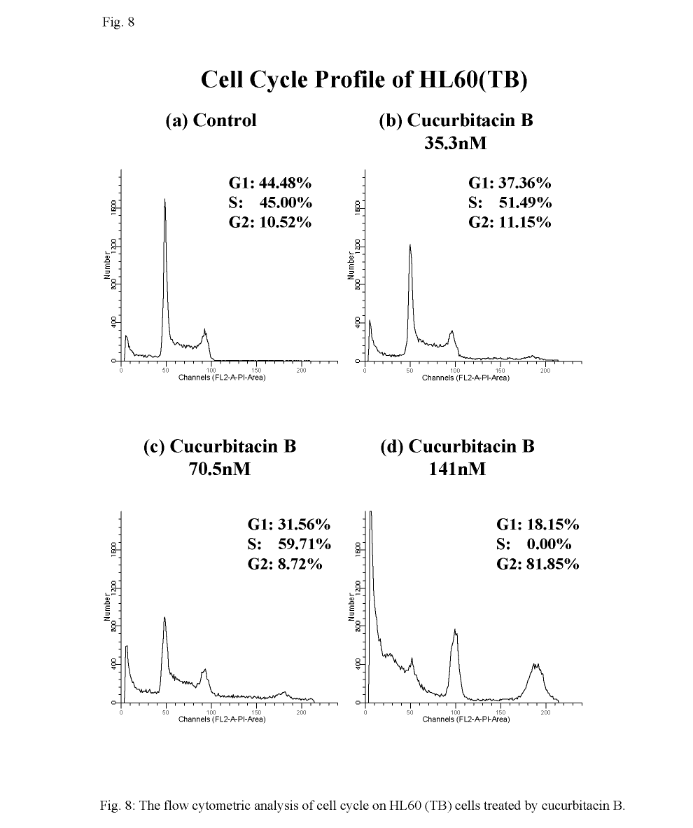

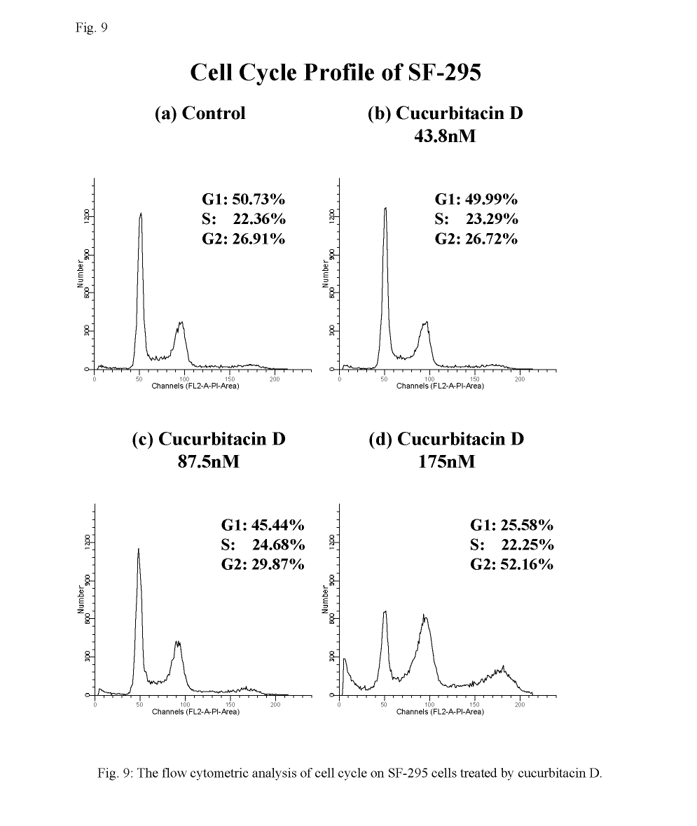

Less G₂/M arrest in the cell lines treated with cucurbitacin B (6 out of 18) were observed (FIG. 6) while half of cell lines (9 out of 18) treated with cucurbitacin D were arrested in G₂/M (FIG. 7). During the 48-hour treatment, endoreduplication (8 n) was observed in most cell lines. A leukemia cell line, HL60 (TB) (FIG. 8), and a CNS cell line, SF-295 (FIG. 9), both displayed G₂/M arrest and endoreduplication with cucurbitacin B and cucurbitacin D treatments, respectively.

### EXAMPLE 5

### Cucurbitacin B and Cucurbitacin D Induced Apoptosis in Human Cancer Cell Lines

Apoptosis is the important mechanism for cell death in normal cells. In cancer cells, this mechanism fails and cells proliferate without control.

Human cancer cell lines from the 59-NCI Cancer Cell Line Panel including leukemia, melanoma and cancers of breast, brain, colon, lung, ovary, prostate and kidney were purchased from the National Institute of Cancer. Cell lines which possess the highest or lowest sensitivity (according to GI₅₀) in response to cucurbitacin B and cucurbitacin D were selected (FIG. 5). They were treated with cucurbitacin B and cucurbitacin D at three different concentrations to elucidate their ability to induce any changes in cell cycle.

Cancer cell lines were maintained in RPMI 1640 supplemented with 5% Fetal Bovine Serum, 2 mM L-glutamine and 1% Penicillin/Streptomycin at 37° C. with 5% CO₂. All chemicals were purchased from Sigma unless specified otherwise.

Cells were inoculated into tissue culture flask at cell concentrations from 50000 to 400,000 cells/ml according to the NCl guideline. The cells were incubated at 37° C. with 5% CO₂ for 24 hours. Cucurbitacin B and cucurbitacin D were added to the cells of final concentrations ranging from 6 to 350 nM (GI₅₀, ½ GI₅₀ and ¼ GI₅₀ of particular cell line) for 48 hours at 37° C. with 5% CO₂. Cells (3×10) were then harvested and stained with 5 μl Annexin V-FITC and 10 μl propidium iodine (Becton Dickinson, Calif., USA) for 15 minutes at room temperature. Ten thousands events were collected by the FACScaliber and the percentage of different cell populations were analyzed by the CellQuest™ software (Becton Dickinson).

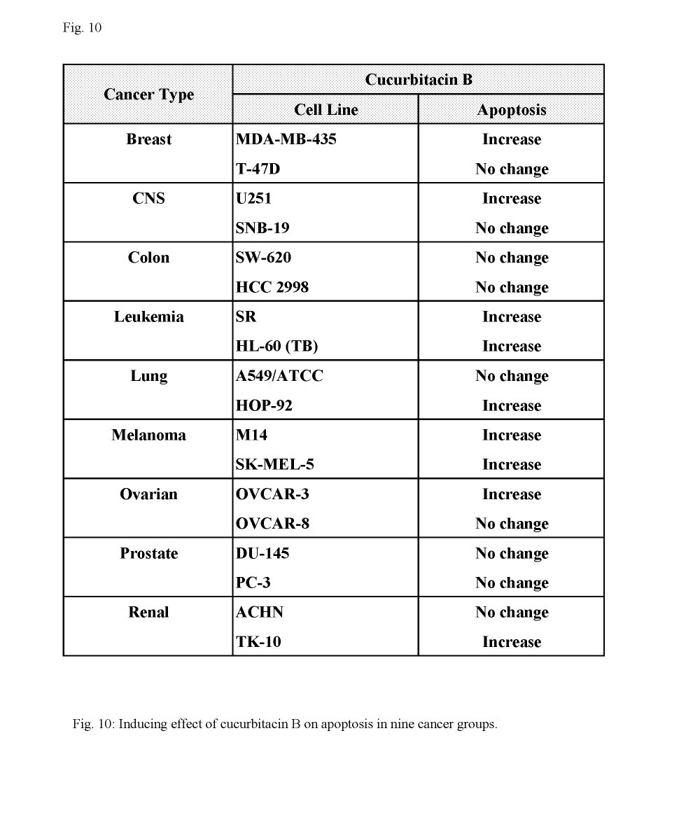

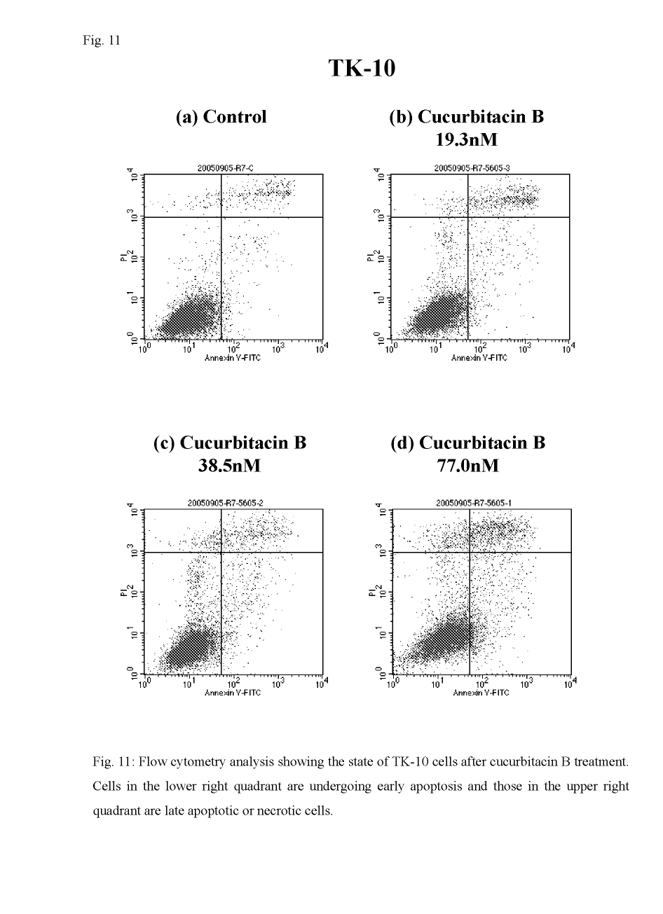

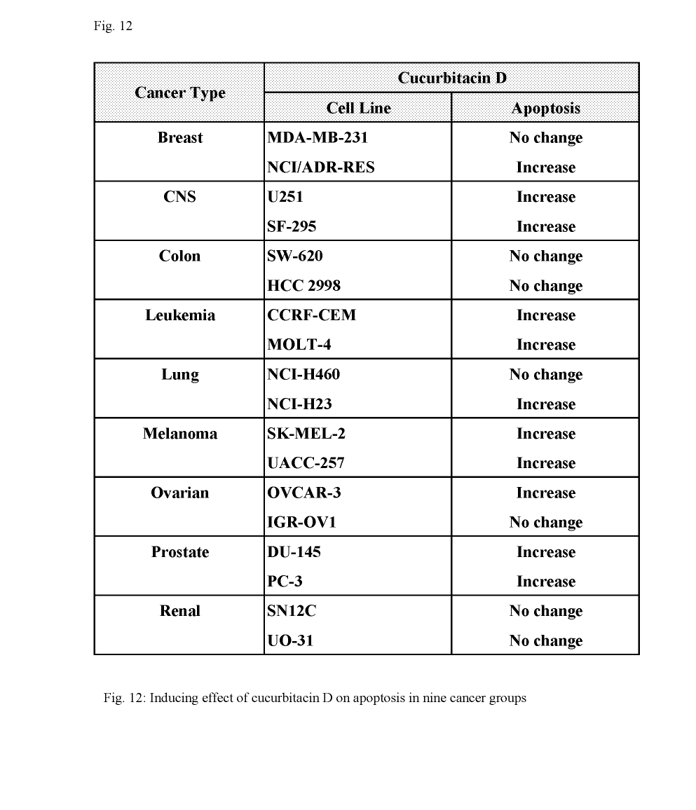

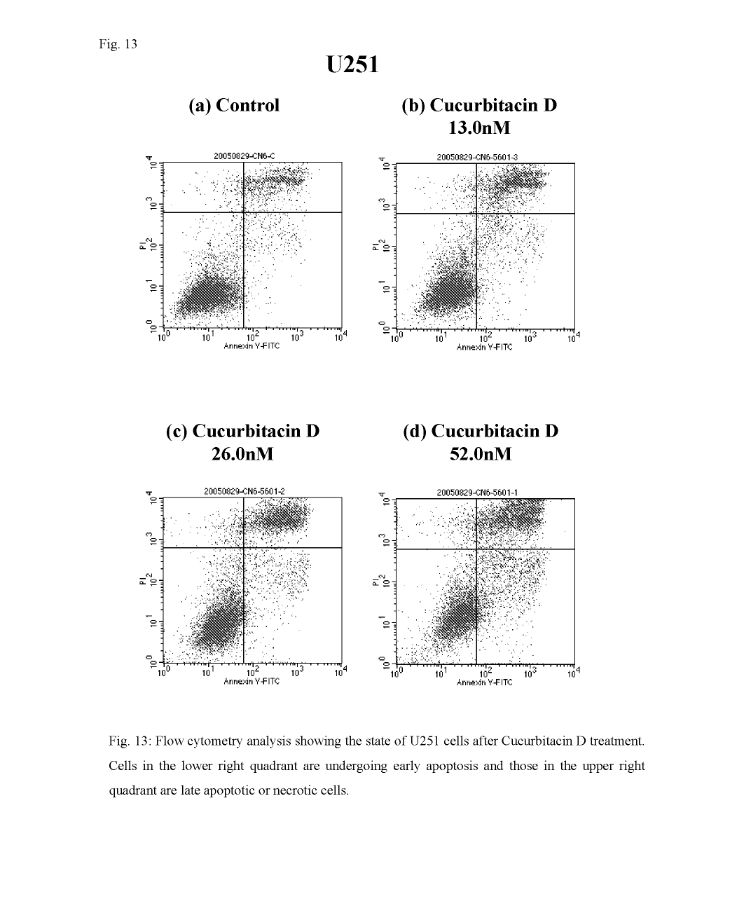

Our results indicated that cucurbitacin B induced apoptosis in 9 out of 18 cell lines (FIGS. 10 and 11) while cucurbitacin D induced apoptosis in 11 out of 18 cell lines (FIGS. 12 and 13). These 2 compounds induced apoptosis in a dose-dependent manner.

### EXAMPLE 6

### Cucurbitacin B and Cucurbitacin D Induced Apoptosis by the Activation of PARP Cell Signaling Pathway—Cell Cycle and Apoptosis

Cucurbitacin B and cucurbitacin D induced apoptosis in human leukemia cell lines, HL 60 and regulated cell cycle via mitogen-activated-protein kinase (MAPK) signaling pathway.

Control cells, as well as cells treated with cucurbitacin B or cucurbitacin D, were harvested and collected by centrifugation. Whole cell extracts were then prepared by lysing the cells using 4% sodium dodecyl sulfate (SDS) gel sample buffer. Cell extracts were boiled for 10 min and chilled on ice, subjected to 12% SDS-polyacrylamide gel electrophoresis, and transferred to a PVDF membrane. Each membrane was cut into to two pieces with one piece incubated at 4^{˜} overnight with antibodies against cell cycle signaling proteins, such as ERK, phosphorlated-ERK, p38, phosphorlated-p38, Cyclin E, Retinoblastoma, phosphalated-Retinoblastoma and c-myc, and apoptotic protein (PARP). β-actin was used as a control for protein loading. All antibodies were obtained from Cell signaling Technologies (USA). Then membranes were incubated at 37^{˜} for 1 h with secondary antibody conjugated with peroxidase, and the signal was detected using chemiluminescence detection reagent. The relative protein level was calculated as the ratio of the optical density of the protein of interest to that of β-actin.

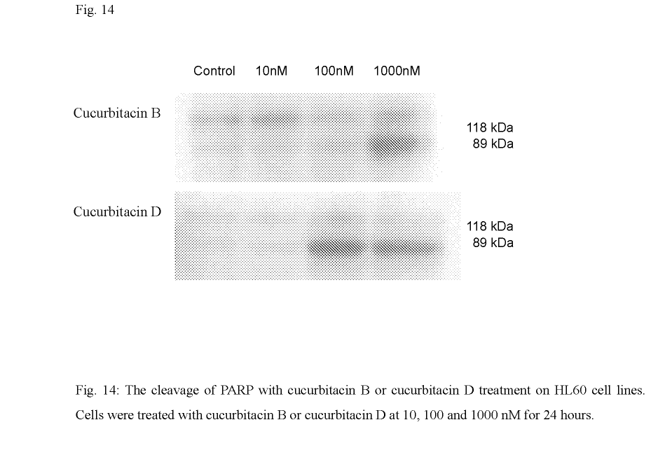

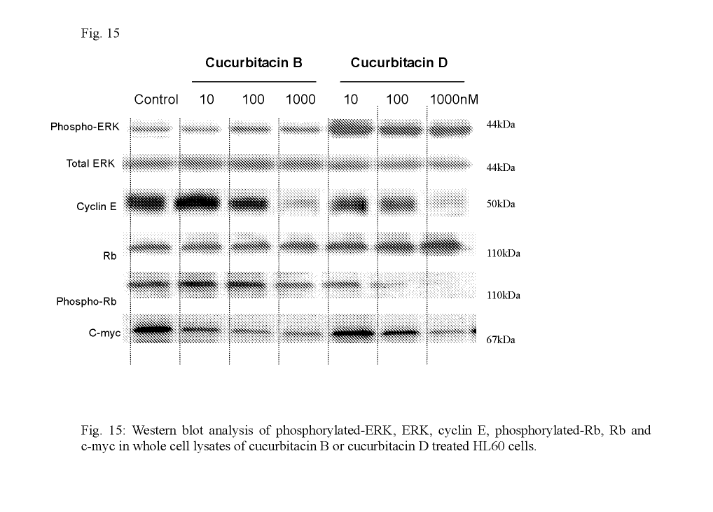

Apoptosis was found upon cucurbitacin B or cucurbitacin 1) incubation. It is demonstrated by the cleavage of PARP, an inducer of apoptosis, when induced with cucurbitacin B or cucurbitacin D treatment (FIG. 14). PARP, a polypeptide of about 118 kDa, will cleave into two fragments of 89 kd and 24 kd when activated and results in the consequence of DNA breakage during apoptosis.

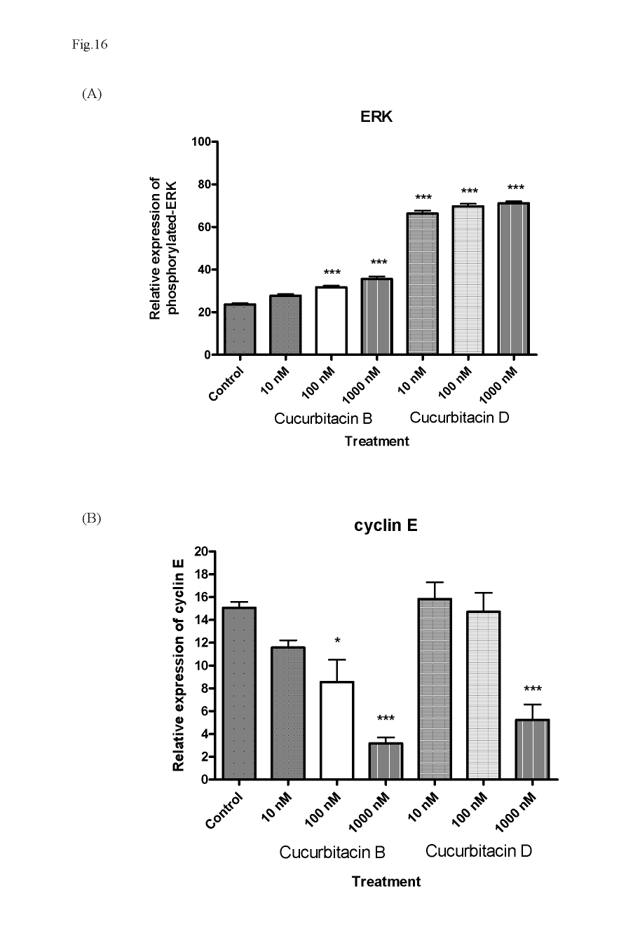

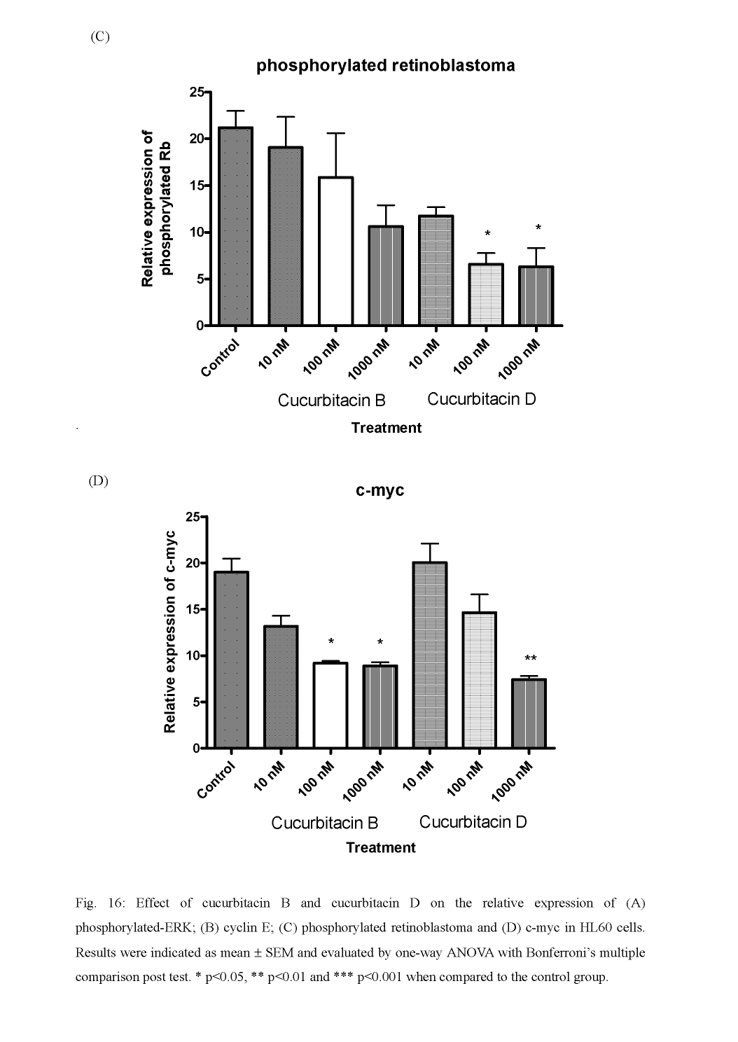

MAPK signaling pathway is a downstream signaling cascade that regulates both cell cycle progression and arrest. It includes four families: the extracellular signal-regulated kinases (ERKs), the c-jun NH₃-terminal kinases/stress activated protein kinase, the p38 MAPKs, and the ERK5 or big MAPKs (Jones et al., 2005). As shown in FIG. 15, our results demonstrated that cell cycle arrest was induced by cucurbitacin B or cucurbitacin D treatment as a consequence of ERK activation, followed by cyclin E down-regulation, inhibition of retinoblastoma's phosphorylation, and ultimately down-regulation of c-myc. C-myc is an onco-protein which is found to be amplified in many types of tumor, including breast, cervical and colon cancers, as well as in squamous cell carcinomas of the head and neck, myeloma, non-Hodgkin's lymphoma, gastric adenocarcinomas and ovarian cancer (Pelengaris et al., 2003). FIG. 16 illustrated that the signaling proteins were regulated in a dose dependent manner with statistical significance.

### PUBLICATIONS

### U.S Patent Documents

U.S. Pat. No. 5,925,356 Subbiah Jul. 9, 1996

### Foreign Patenet Documents

WO 02/078617 Sebti, et al. Mar. 28, 2002

### Other Publications

Ahmad, A., Khan, K. A., Ahmad, V. U., Qazi, S., 1986 Antibacterial activity of juliflorine isolated from Prosopis juliflora. Planta Med 4: 285-288.
Arisawa, M., Pezzuto, J. M., Kinghorn, A. D., Cordell, G. A., Farnsworth, N. R., 1984 Plant anticancer agents. XXX: Cucurbitacins from Ipomopsis aggregata (Plolemoniaceae). J Pharm Sci. 73(3): 411-413.
Barbercheck, M. E. and Wang, J., 1996 Effect of cucurbitacin D on in vitro growth of Xenorhabdus and Photorhabdus spp., symbiotic bacteria of entomopathogenic nematodes. J Invertebr Pathol. 68(2): 141-145.
Bean, M. F., Antoun, M., Abramson, D., Chang, C. J., McLaughlin, J. L., Cassady, J. M., 1985 Cucurbitacin B and isocucurbitacin B: cytotoxic components of Helicteres isora. J Nat. Prod. 48(3): 500.
Beutler, J. A., McCall, K. L., Herbert, K., Herald, D. L., Pettit, G. R., Johnson, T., Shoemaker, R. H., Boyd, M. R., 2000 Novel cytotoxic diterpenes from Casearia arborea. J Nat. Prod. 63(5): 657-661.
Chen, J. C., Chiu, M. H., Nie, R. L., Cordell, G. A., Qiu, S. X., 2005 Cucurbitacins and cucurbitane glycosides: structures and biological activities. Nat Prod Rep. 22(3): 386-399.
Clericuzio, M., Mella, M., Vita-Finzi, P., Zema, M., Vidari, G., 2004 Cucurbitane triterpenoids from Leucopaxillus gentianeus. J Nat Prod. 67(11): 1823-1828.
Douglas, R. S., Tarshis, A. D., Pletcher, C. H. Jr., Nowell, P. C., Moore, J. S., 1995 A simplified method for the coordinate examination of apoptosis and surface phenotype of murine lymphocytes. J Immunol Methods. 188: 219-228.
Duncan, K. L., Duncan, M. D., Alley, M. C., Sausville, E. A., 1996 Cucurbitacin E-induced disruption of the actin and vimentin cytoskeleton in prostate carcinoma cells. Biochem Pharmacol. 52(10): 1553-1560.
Edery, H., Schatzberg-Porath, G., Gitter, S., 1961 Pharmaco-dynamic activity of elatericin (cucurbitacin D). Arch Int Pharmacodyn Ther. 130: 315-335.
Elledge, S. J., 1996 Cell cycle checkpoints: preventing an identity crisis. Science. 274(5293): 1664-1672.
Fang, X., Phoebe, C. H., Pezzuto, J. M. Jr., Fong, H. H., Farnsworth, N. R., Yellin, B., Hecht, S. M., 1984 Plant anticancer agents, XXXIV. Cucurbitacins from Elaeocarpus dolichostylus. J Nat Prod. 47(6): 988-993.
Fuller, R. W., Cardellina, J. H., Cragg, G. M., Boyd, M. R., 1994 Cucurbitacins: differential cytotoxicity, dereplication and first isolation from Gonystylus keithii. J Nat. Prod. 57(10): 1442-1445.
Johnson, L. N., De Moliner, E., Brown, N. R., Song, H., Barford, D., Enclicott, J. A., Noble, M. E., 2002 Structural studies with inhibitors of the cell cycle regulatory kinase cyclin-dependent protein kinase 2. Pharmacol Ther. 93(2-3): 113-124.
Jones, N. C., Tyner, K. J., Nibarger, L., Stanley, H. M., Cornelison, D. D., Fedorov, Y. V., Olwin, B. B., 2005 The p38α/β MAPK functions as a molecular switch to activate the quiescent satellite cell. J. Cell Biol. 169(1): 105-116.
Kalejta, R. F., Shenk, T., Beavis, A. J., 1997 Use of a membrane-localized green fluorescent protein allows simultaneous identification of transfected cell s and cell cycle analysis by flow cytometry. Cytometry. 29:286-291.
Konoshima, T., Takasaki, M., Kozuka, M., Nagao, T., Okabe, H., Irino, N., Nakasumi, T., Tokuda, H., Nishino, H., 1995 Inhibitory effects of cucurbitane triterpenoids on Epstein-Barr virus activation and two-stage carcinogenesis of skin tumor. II. Biol Pharm Bull. 18(2): 284-287.
Dinan, L., Whiting, P., Girault, J. P., Lafont, R., Dhadialla, T. S., Cress, D. E., Mugat, B., Antoniewski, C., Lepesant, J. A., 1997 Cucurbitacins are insect steroid hormone antagonists acting at the ecdysteroid receptor. Biochem J 327: 643-650.
Morgan, D. O., 1997 Cyclin-dependent kinases: engines, clocks, and microprocessors. Annu Rev Cell Dev Biol. 13: 261-291.
Musza, L. L., Speight, P., McElhiney, S., Barrow, C. J., Gillum, A. M., Cooper, R., Killar, L. M., 1994 Cucurbitacins, cell adhesion inhibitors from Conobea scoparioides. J Nat Prod. 57(11): 1498-1502.
Oberlies, N. H., Burgess, J. P., Navarro, H. A., Pinos, R. E., Soejarto, D. D., Farnsworth, N. R., Kinghorn, A. D., Wani, M. C., Wall, M. E., 2001 Bioactive constituents of the roots of Licania intrapetiolaris. J Nat Prod. 64(4): 497-501.
Pelengaris, S., Khan, M., 2003 The many faces of c-MYC. Arch Biochem Biophys. 416(2): 129-136.
Rodriguez, N., Vasquez, Y., Hussein, A. A., Coley, P. D., Solis, P. N., Gupta, M. P., 2003 Cytotoxic cucurbitacin constituents from Sloanea zuliaensis. J Nat. Prod. 66(11): 1515-1516.
Setzer, W. N., Setzer, M. C., 2003 Plant-derived triterpenoids as potential antineoplastic agents. Mini Rev Med. Chem. 3(6): 540-556.
Skehan, P., Storeng, R., Scudiero, D., Monks, A., McMahon, J., Vistica, D., Warren, J. T., Bokesch, H., Kenney, S., Boyd, M. R., 1990 New calorimetric cytotoxicity assay for anticancer-drug screening. J Natl Cancer Inst. 82(13): 1107-12.
Smit, H. F., van den Berg, A. J., Kroes, B. H., Beukelman, C. J., Quarles van Ufford, H. C., van Dijk, H., Labadie, R. P., 2000 Inhibition of T-lymphocyte proliferation by cucurbitacins from Picrorhiza scrophulariaeflora. J Nat Prod. 63(9): 1300-1302.
Stuppner, H. and Wagner, H. 1989 New cucurbitacin glycosides from Picrorhiza kurroa. Planta Med 55: 559-563.
Vermes, I., Hananen, C., Steffens-Nakken, H., Reutelingsperger, C., 1995 A novel assay for apoptosis. Flow cytometric detection of phosphatidylserine expression on early apoptotic cells using fluorescein labeled Annexin V. J Immunol Meth. 184: 39-51.
Yesilada, E., Tanaka, S., Sezik, E., Tabata, M., 1988. Isolation of an anti-inflammatory principle from the fruit juice of Ecballium elaterium. J Nat Prod. 51(3): 504-508.
Yesilada, E., Tanaka, S., Sezik, E., Tabata, M., 1988. Isolation of an anti-inflammatory principle from the fruit juice of Ecballium elaterium. J Nat Prod. 51(3): 504-508.

What is claimed is:

1. A method of inducing a cytostatic effect on cancer cells in a subject, comprising:
administering an effective amount of an isolated cucurbitacin to said subject, to induce said cytostatic effect in said cancer cells.

2. The method of claim 1, wherein said cucurbitacin comprises cucurbitacin B, and said administering an effective amount comprises contacting said cancer cells with cucurbitacin B at a concentration of 5.8 nM to 164 nM.

3. The method of claim 1, wherein said cucurbitacin comprises cucurbitacin D, and said administering an effective amount comprises contacting said cancer cells with cucurbitacin D at a concentration of 14 nM to 324 nM.

4. The method of claim 1, wherein said cancer cells are selected from the group consisting of leukemia cells, melanoma cells, breast cancer cells, brain cancer cells, colon cancer cells, lung cancer cells, ovary cancer cells, renal cancer cells, prostate cancer cells and kidney cancer cells.

5. The method of claim 4, wherein said cucurbitacin comprises cucurbitacin B, said cancer cells are prostate cancer cells, and said administering an effective amount comprises contacting said cancer cells with cucurbitacin B at a concentration of 17 nM.

6. The method of claim 4, wherein said cucurbitacin comprises cucurbitacin D, said cancer cells are melanoma cells, and said administering an effective amount comprises contacting said cancer cells with cucurbitacin D at a concentration of 60 nM.

7. A method of inducing cell cycle arrest in cancer cells in a subject, comprising:
activating the MAPK signaling pathway by administering an effective amount of an isolated cucurbitacin to said subject, to induce cell cycle arrest in said cancer cells.

8. The method of claim 7, wherein said cancer cells are selected from the group consisting of leukemia cells, melanoma cells, breast cancer cells, brain cancer cells, colon cancer cells, lung cancer cells, ovary cancer cells, renal cancer cells, prostate cancer cells and kidney cancer cells.

9. The method of claim 8, wherein said cucurbitacin comprises cucurbitacin B, and said cancer cells are leukemia cells.

10. The method of claim 8, wherein said cucurbitacin comprises cucurbitacin D, and said cancer cells are brain cancer cells.

11. A method of inducing apoptosis in cancer cells in a subject, comprising:
activating the PARP pathway by administering from 6 nM to 350 nM of an isolated cucurbitacin to said subject, to induce apoptosis in said cancer cells.

12. The method of claim 11, wherein said cancer cells are selected from the group consisting of leukemia cells, melanoma cells, breast cancer cells, brain cancer cells, colon cancer cells, lung cancer cells, ovary cancer cells, renal cancer cells, prostate cancer cells and kidney cancer cells.

13. The method of claim 12, wherein said cucurbitacin comprises cucurbitacin B, said cancer cells are colon cancer cells, and said administering comprises contacting said cancer cells with cucurbitacin B at a concentration of 5.8 nM to 64.5 nM.

14. The method of claim 12, wherein said cucurbitacin comprises cucurbitacin B, said cancer cells are breast cancer cells, and said administering comprises contacting said cancer cells with cucurbitacin B at a concentration of 18.7 nM to 110.7 nM.

15. The method of claim 12, wherein said cucurbitacin comprises cucurbitacin D, said cancer cells are ovary cancer cells, and said administering comprises contacting said cancer cells with cucurbitacin D at a concentration of 90.6 nM to 154.4 nM.

16. The method of claim 12, wherein said cucurbitacin comprises cucurbitacin D, said cancer cells are prostate cancer cells, and said administering comprises contacting said cancer cells with cucurbitacin D at a concentration of 92.3 nM to 105.7 nM.
